(19) United States
(12) Patent Application Publication
(10) Pub. No.: US 2021/0022340 A1
(43) Pub. Date: Jan. 28, 2021

(54) USE OF PYRIDINE UREA COMPOUND HAVING SNAIL-KILLING ACTIVITY

(71) Applicant: THE NATIONAL INSTITUTE OF PARASITIC DISEASES, CHINESE CENTER FOR DISEASE CONTROL AND PREVENTION, Shanghai (CN)

(72) Inventors: Liping DUAN, Shanghai (CN); Shizhu LI, Shanghai (CN); Weisi WANG, Shanghai (CN); Haobing ZHANG, Shanghai (CN); Chunli CAO, Shanghai (CN); Junmin YAO, Shanghai (CN); Zhiqiang QIN, Shanghai (CN); Yi TAO, Shanghai (CN)

(21) Appl. No.: 16/755,036

(22) PCT Filed: Aug. 30, 2018
(86) PCT No.: PCT/CN2018/103256

§ 371 Date: Apr. 9, 2020

(30) Foreign Application Priority Data
Oct. 11, 2017 (CN) 201710941579.5

(51) Int. Cl. A01N 43/40

(52) U.S. Cl. CPC A01N 43/40

(57) ABSTRACT

The present invention relates to use of a pyridine urea compound having snail-killing activities, and relates to a method for preparing the pyridine urea compound. In particular, the present invention discloses a compound having the structure as shown in formula (I), an optical isomer thereof, a racemate thereof, a solvate thereof, or a pharmaceutically acceptable salt thereof, the compound having a significant killing effect on various snails as parasitic disease vectors and low toxicity to non-target organism fish.

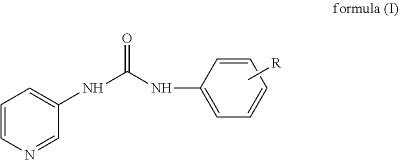

### CROSS-REFERENCE TO RELATED APPLICATIONS

This application claims the benefit of PCT/CN 2018/103256, filed on Aug. 30, 2018, the content of which are hereby incorporated by reference in their entirety

### FIELD OF THE PRESENT INVENTION

The present invention relates to the field of chemical medicine, particularly relates to use of pyridine urea compound having snail-killing activity.

### BACKGROUND OF THE PRESENT INVENTION

Massive reproduction of snails usually brings serious hazards, such as making pond water become shallow quickly, and the amount of plankton greatly reduced. At the same time, snails consume dissolved oxygen content in the water, the growth of fish seed is affected. For another example, some snails are carriers of pathogens. For example, Lymnaea is intermediate hosts of troglobic trematode and Dipostomum, lake snails are intermediate hosts of Pleurogenidae, and Oncomelania hupensis are the only intermediate hosts of Schistosoma cercariae. Once humans and livestock contact with Oncomelania hupensis which carries with Schistosoma cercariae. they would infect schistosomiasis. China is currently threatened by schistosomiasis with a population of 60 million and the number of infected people is 370,000. Therefore, killing Oncomelania hupensis or killing cercariae in Oncomelania hupensis becomes an effective measure to prevent and control schistosomiasis.

In addition, after an alien invasive organism Pomacea canaliculata, as a vector of Angiostrongylus cantonensis, was introduced into China in 1981. Pomacea canaliculata has been cultivated throughout the country of China and spread rapidly over rivers, lakes and fields due to large size, wide appetite, strong adaptability, rapid growth and reproduction, and high yield. Pomacea canaliculatae, which have large appetite and intake with a variety of foods, can destroy the food crops, vegetables and aquatic crops, and have become harmful animals in Guangdong, Guangxi, Fujian, Yunnan, Zhejiang, Shanghai, Jiangsu and etc. In addition, Pomacea canaliculatae is an intermediate host of zoonotic Angiostrongylus cantonensis which can easily cause health problems to surrounding residents.

To date, chemotherapy is still one of the main means to control parasitic diseases mediated by snails. For example, niclosamide is a type of molluscicide with the best Oncomelania hupensis-killing activity at the present time, as well as significant cercariae-killing activity it has. It is also the only molluscicide recommended by the World Health Organization (WHO). However, the disadvantages of niclosamide are still obvious, that is, the toxicity to non-target organisms, especially fish, is high which limits its use. At present, the research on this kind of compounds is focused on improvement of the formulation of niclosamide, such as niclosamide suspension. These can solve the problem of its solubility, but cannot fundamentally solve its problem of toxicity to fish.

Therefore, research and development of novel, high-efficiency, and low-toxic molluscicide has become an urgent technical problem to be solved in the field.

### SUMMARY OF THE PRESENT INVENTION

An object of the present invention is to provide a novel, high-efficiency, and low-toxic molluscicide for preventing parasites and vectors of parasites including the alien invasive organism Pomacea canaliculata and etc.

Another object of the present invention is to provide protection for people susceptible to schistosomiasis and Angiostrongylus cantonensis from the dangers of schistosomiasis and Angiostrongylus cantonensis.

A first aspect of the present invention provides a compound having a structure of Formula (I), or an optical isomer, or a racemate thereof, or a solvate thereof, or a pharmaceutically acceptable salt thereof:

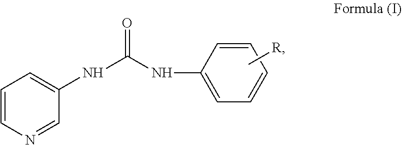

in formula (I), substituent R means being substituted by one or more substituents selected from the group consisting of halogen, nitro, hydroxyl, cyano, C₁-C₆ alkyl, C₂-C₆ alkenyl, C₂-C₆ alkynyl, C₁-C₆ alkoxy, and C₃-C₆ cycloalkyl.
Preferably, substituent R is selected from the group consisting of p-methoxy, fluorine, chlorine, bromine, trifluoromethyl, methyl, nitro and hydrogen.

A second aspect of the present invention provides an agricultural composition including a compound (a) according to the first aspect of the present invention or a pharmaceutically acceptable salt thereof, and an agrochemically acceptable carrier or excipient.

In another preferred embodiment, the weight of compound (a) present in the agricultural composition is from 0.0001% to 99.99%, preferably from 0.001% to 99.9%. more preferably from 0.01% to 99%.

In another preferred embodiment, the agricultural composition further includes other molloscicidc; the other molluscicide is commercially available.

In another preferred embodiment, the other molluscicide is selected from the group consisting of niclosamide, Rongbao, Rongya, tee tree seed and other commercially available plant molluscicides.

A second aspect of the present invention provides a use of the compound according to the first aspect of the present invention or the composition according to the second aspect of the present invention for preventing parasites and or killing a parasitic vector, or preparing medicine for preventing the parasites and or killing the parasitic vector.

In another preferred embodiment, the parasite includes nematode, tapeworm, and trematode.

In another preferred embodiment, the parasitic vector includes Oncomelania hupensis, Pomacea canaliculata, and snail.

A fourth aspect of the present invention provides a method for preventing parasitic diseases. Wherein the compound according to the first aspect of the present invention or the agricultural composition according to the second aspect of the present invention is applied to a parasitic vector or an environment suffering from the disaster of the vector (such as soil, waters, and etc.).

In another preferred embodiment, the compound or the agricultural composition is applied at a concentration from 0.02 mg/L to 5 mg/L; preferably from 0.01 mg/L to 1 mg/L; more preferably from 0.1 mg/L to 0.5 mg/L.

A fifth aspect of the present invention provides a use of a compound of formula (I), or an optical isomer, or a racemate thereof, or a solvate thereof, or a pharmaceutically acceptable salt thereof, for the preparation of an agricultural composition or a formulation, the agricultural composition or formulation is for (a) preventing parasites; and or (b) killing a vector for the parasites, wherein the parasitic vector is various snails.

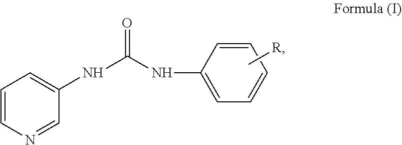

in formula (I), substituent R is a group selected from the group consisting of p-methoxy, fluorine, chlorine, bromine, trifluoromethyl, methyl, nitro and hydrogen.

In another preferred embodiment, the parasite includes nematode, tapeworm, and trematode.

In another preferred embodiment, the parasitic vector includes Oncomelania hupensis, Pomacea canaliculata, and snail.

A sixth aspect of the present invention further provides an agricultural composition comprising (a) an active component including the compound according to the first aspect of the present invention, an optical isomer thereof, a racemate thereof, a solvate thereof, or a pharmaceutically acceptable salt thereof, and (b) an agrochemically acceptable carrier or excipient.

In another preferred embodiment, the agricultural composition contains from 0.0001 wt % to 99.99 wt %, preferably from 0.001 wt % to 99.9 wt %, more preferably 0.01-99 wt % of the compound of formula (I), or an optical isomer, or racemate thereof, or a solvate thereof, or a pharmaceutically acceptable salt thereof, based on the total weight of the agricultural composition.

In another preferred embodiment, the agricultural composition further includes other molluscicide, the other molluscicide is commercially available.

In another preferred embodiment, the other molluscicide is selected from the group consisting of niclosamide, Rongbao, Rongya, tee tree seed and other commercially available plant molluscicides.

A seventh aspect of the present invention further provides a use of the agricultural composition according to the sixth aspect of the present invention for preventing parasites and/or killing a parasitic vector, or preparing medicine for preventing the parasites and or killing the parasitic vector.

An eighth aspect of the present invention provides a method for preventing parasitic diseases. Wherein the compound according to the first aspect of the present invention, an optical isomer thereof, a racemate thereof, a solvate thereof, or a pharmaceutically acceptable salt thereof, or the agricultural composition according to the sixth aspect of the present invention is applied to a parasitic vector or an environment suffering from the disaster of the vector.

In another preferred embodiment, the compound according to the first aspect of the present invention, an optical isomer thereof, a racemate thereof, a solvate thereof, or a pharmaceutically acceptable salt thereof, or the agricultural composition according to the sixth aspect of the present invention application concentration is applied at a concentration from 0.02 mg/L to 5 mg/L; preferably from 0.01 mg/L to 1 mg/L; more preferably from 0.1 mg/L to 0.5 mg/L.

It should be understood that the above technical features of the present invention and the technical features specifically described in the following (such as the embodiments) may be combined with each other to form a new or preferred technical solution within the scope of the present invention. Due to space limitations, there won't be repeated description again.

### DETAILED DESCRIPTION OF THE PREFERRED EMBODIMENT

Through long-term and in-depth research, the inventors have discovered molluscicide with a novel structure, which has advantages of significantly improved snail-killing activities and cercariae-killing activity, and has low toxicity to non-target organisms. On this basis, the inventors have completed the present invention.

Group Definition

As used herein, the term “C₁-C₆ hydrocarbyl” refers to an alkyl group having from 1 to 6 carbon atoms, alkenyl group, alkynyl group, cycloalkyl group, cycloalkenyl group, aryl group and the like which contains only carbon, hydrogen or unsaturatcd group. Preferred is an alkyl group, a cycloalkyl group, an alkenyl group or an alkynyl group.

As used herein, the term “C₁-C₆ alkyl” refers to a straight or branched alkyl group having from 1 to 6 carbon atoms, such as methyl group, ethyl group, propyl group, isopropyl group, butyl group, isobutyl group, sec-butyl group, tert-butyl group, or the like.

As used herein, the term “C₂-C₆ alkenyl” refers to a straight or branched alkenyl group having from 2 to 6 carbon atoms, such as vinyl group, allyl group. 1-propenyl group, isopropenyl group, 1-butenyl group, 2-butenyl group, or the like.

As used herein, the term “C₂-C₆ alkynyl” refers to a straight or branched alkynyl group having from 2 to 6 carbon atoms, such as ethynyl group, propynyl group, and the like.

As used herein, the term “C₃-C₆ cycloalkyl” refers to cyclopropyl group, cyclobutyl group, cyclopentyl group, cyclohexyl group, or cycloheptyl group, and the like.

The term “C₁-C₆ alkoxy” refers to a straight or branched alkoxy group having from 1 to 6 carbon atoms, such as methoxy group, ethoxy group, propoxy group, isopropoxy group, butoxy group, isobutoxy group, sec-butoxy group, tert-butoxy group, or the like.

The term “halogen” refers to fluorine, chlorine, bromine, or iodine. The term “halo” refers to a group substituted with one or more of the above halogen atoms, such as trifluoromethyl group, pentafluoroethyl group, or the like.

“Substituent” in the present invention means substituted by one or more conventional substituents, for example, selected from the group consisting of halogen, nitro, hydroxyl, cyano. C₁-C₆ alkyl, C₂-C₆ alkenyl, C₂-C₆ alkynyl, C₁-C₆ alkoxy, and C₃-C₆ cycloalkyl.

Antiparasitic activity of the active substance according to the present invention

The term “active substance of the present invention” or “active compound of the present invention” refers to a compound of formula (I) according to the present invention. The active substance of the present invention can effectively control and destroy parasites and their vector, and the like, and has significant activity killing vectors of parasites and anti-parasite activity.

Examples of parasites include but are not limited to:

Trematode: such as Clonorchis sinensis, Fasciolopsis buski, Fasciola hepatica, Paragonimiasis, Schistosoma japonicum, S. haematobium, S. mansoni, S. inter calatum, S. mekongi, S. malayensis.

Tapeworm: such as Spirometra mansoni, Diphyllobothrium latum, Taenia solium, Taenia saginata, Taenia saginata asiatica, Hymenolepis nana, Hymenolepis diminuta, Echinococcus granulosus, Echinococcus multilocularis, Dipylidium caninum.

Nematode, such as Ascaris lumbricoides, Angiostrongylus cantonensis, Trichuris trichiura, Enterobius vermicularis, Ancylostoma duodenale, Necator americanus, Strongyloides stercoralis, Trichinella spiralis, Wuchereria bancrofti, Brugia malayi.

Examples of parasitic vectors include but are not limited to snails, such as Oncomelania hupensis, Pomacea canaliculata, and snail.

The “anti-parasite medicine” or “parasite-preventing medicine” according to the present invention is a collective term for substances having the functions of preventing all the parasites mentioned above and the transmission vector.

The active compounds of the present invention are particularly effective against trematodes such as Schistosoma japonicum, tapeworms (such as Echinococcus granulosus and Echinococcus multilocularis). Oncomelania hupensis, and etc.

Molluscicide composition containing the active substance of the present invention and modification of its formulation.

The active substance of the present invention can be prepared into a molluscicide composition by a conventional method. These active compounds can be made into conventional formulations such as solutions, emulsions, suspensions, powders, foams, pastes, granules.

These formulations can be produced by known methods, for example, by mixing an active compound with an extender which is liquid, liquefied gas, solid diluent, or carrier, as well as optionally surfactant such as emulsifier, dispersant, foam former. For example, when using water as the extender, organic solvents can also be used as a coagent.

It is basically suitable for a liquid solvent to be used as a diluent or carrier, for example aromatic hydrocarbons such as xylene, toluene, or alkyl naphthalene, chlorinated aromatic hydrocarbons or aliphatic hydrocarbons such as chlorobenzene, vinyl chloride or dichloromethane, aliphatic hydrocarbons such as cyclohexane or paraffin (such as mineral oil fractions); alcohols such as ethanol or ethylene glycol, and their ethers and lipids; ketones such as acetone, methyl ethyl ketone, methyl isobutyl ketone or cyclohexanone; or polar solvents that are not commonly used such as dimethylformamide and dimethyl sulfoxide, and water.

In the case of the diluent or carrier of liquefied gas, it refers to a liquid that will become a gas at normal temperature and pressure, for example aerosol propellants such as halogenated hydrocarbons, butane, propane, nitrogen, and carbon dioxide.

The solid carrier can be ground natural minerals such as kaolin, clay, talc, quart, activated clay, montmorillonite or diatomaceous earth, and ground synthetic minerals such as highly dispersed silic acid, alumina and silicates. The solid carriers for granules are crushed and graded natural zircon such as calcite, marble, pumice, sepiolite, and dolomite, and granules synthesized by inorganic and organic coarse powders such as sawdust, and organic materials such as sawdust, coconut shell, Corn cobs and granules of tobacco stems.

Nonionic and anionic emulsification can be used as emulsifiers and/or foam formers. For example, polyoxyethylene-fatty acid esters, polyoxyethylene-fatty alcohol ethers, alkylaryl polyethylene glycol ethers, alkyl sulfonates, alkyl sulfates, aryl sulfonates, and Albumin hydrolysate. Dispersants includes lignin sulfite waste liquid and methyl cellulose.

Binders such as carboxymethyl cellulose and natural and synthetic polymers in the form of powders, granules or emulsions may be used in the formulations, such as gum arabic, polyvinyl alcohol and polyvinyl acetate.

Colorants may be used, for example, inorganic dyes such as iron oxide, cobalt oxide, and Prussian blue: organic dyes such as azo dyes or metal phthalocyanine dyes; and trace nutrients such as Iron, Manganese, Boron, Copper, Cobalt, Aluminum and Zinc.

The molluscicide composition generally contains from 0.001% to 99.99% by weight, preferably from 0.01% to 99.9% by weight, and more preferably from 0.05% to 90% by weight of the active compound of the present invention.

These active compounds of the present invention and other active compounds may be made into mixture present in their commercial formulations or in dosage forms prepared from these formulations.

These other active compounds may be other molluscicides commercially available such as niclosamide. Rongbao, Rongya, tee tree seeds, and other commercially available plant molluscicides. Wherein Rongbao and Rongya are two types of molluscicide. Rongbao is solid, and the main component is calcium cyanamide Rongya is liquid and the content is 25%, and the main component is monocyandiamide.

These other active compounds may also be insecticides, baits, bactericides, acaricides, nematicides, fungicides, growth control agents, and the like. Insecticides include such as phosphates, urethanes, pyrethroids, chlorinated hydrocarbons, benzoylureas, nereistoxin, and substances produced by microorganisms such as avermectin.

In addition, the active compounds of the present invention and a synergist may also be made into mixture present in their commercial formulations or in dosage forms prepared from these formulations. The synergist is a compound that enhances the action of the active compounds. If the active compound itself is active, the synergist is not necessary to be added.

The active compound of the dosage form prepared from a commercial formulation contains a concentration varied within a wide range. The concentration of the active compound in the dosage form may be from 0.0000001% to 100% (g/v). preferably between 0.0001% and 1%.

The present invention mainly includes the following advantages:

(1) The molluscicide of the present invention has significant snail-killing activity and cercariae-killing activity, and has low toxicity to non-target organisms.

(2) The preparation method of the molluscicide of the present invention is simple, and the operability is good.

The present invention will be further described below with reference to specific embodiments. It should be understood that these examples are only used to illustrate the present invention and not to limit the scope of the present invention. The experimental methods without specific conditions in the following embodiments are generally based on conventional conditions or conditions recommended by the manufacturer. Percentages and parts are noted by weight unless stated otherwise.

### Embodiment (I): Preparation Method (I) of Pyridine Urea Compounds is Applied to Compound 1 to Compound 17

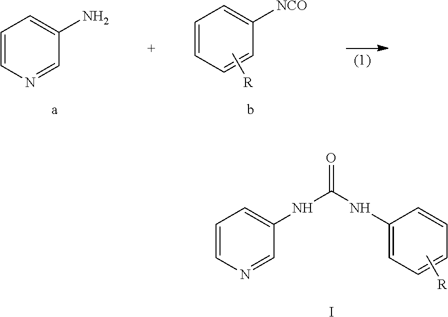

At room temperature, add compound a (10 mmol) and compound b (12 mmol) to anhydrous acetonitrile (40 mL), and triethylamine (about 1 ml) was added dropwise with heating, and heated to reflux for 6 hours, then cooled and filter. After washing with water, the residue was dried, and used hot water to wash die residue three times with, then recrystallized in ethanol to obtain a white solid I.

### EXAMPLE 1

Compound 1 shown below was prepared according to the method of Embodiment (I) which is substituted compound b by using

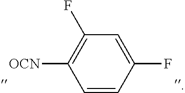

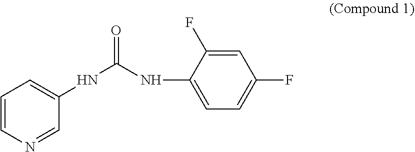 [SMILES: O=C(Nc1cccnc1)Nc1ccc(F)cc1F]

The product represented as Compound 1 of Example 1 is white solid, and yield is 81%. mp: 191.5° C.-193.8° C. ¹H NMR (400 MHz, DMSO) δ 8.60 (d, J=2.5 Hz, ¹H, Ar—H), 8.20 (dd, J=4.7, 1.4 Hz, ¹H, Ar—H), 8.04 (td, J=9.2, 6.2 Hz, ¹H, Ar—H), 7.95 (ddd, J=8.3, 2.6. 1.5 Hz, ¹H, Ar—H), 7.34-7.29 (m, ²H, Ar—H), 7.08-7.03 (m, ¹H, Ar—H). HRMS-ESI m/z [M+H]+calcd for C₁₂H₉F₂N₃O: 250.0714, found: 250.0776.

### EXAMPLE 2

Compound 2 shown below was prepared according to the method of Embodiment (I) which is substituted compound b by using

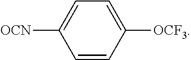

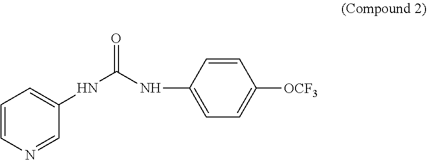 [SMILES: O=C(Nc1ccc(OC(F)(F)F)cc1)Nc1cccnc1]

The product represented as Compound 2 of Example 2 is white solid, and yield is 84%. mp: 178.3° C.-178.7° C. ¹NMR (400 MHz, DMSO) δ 9.08 (br, ²H, NH), 8.62 (d, J=2.5 Hz, ¹H, Ar—H), 8.20 (dd, J=4.7, 1.4 Hz, ¹H, Ar—H), 7.94 (ddd, J=8.3, 2.5, 1.5 Hz, ¹H, Ar—H), 7.59-7.55 (m, ²H, Ar—H), 7.34-7.29 (m, ³H, Ar—H). HRMS-ESI m/z [M+H]+calcd for C₁₃H₁₀F₃N₃O₂: 298.0725, found: 298.0748.

### EXAMPLE 3

Compound 3 shown below was prepared according to the method of Embodiment (I) which is substituted compound b by using R—Cl

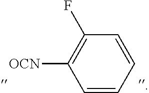

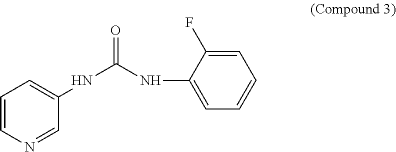 [SMILES: O=C(Nc1cccnc1)Nc1ccccc1F]

The product represented as Compound 3 of Example 3 white solid, and yield is 75%. mp: 183.4° C.-185.0° C. ¹H NMR (400 MHz, DMSO) δ 8.66 (d, J=2.5 Hz, ¹H, Ar—H), 8.26 (dd, J=4.7, 1.4 Hz, ¹H, Ar—H), 8.18 (td, J=8.2, 1.6 Hz, ¹H, Ar—H), 8.02 (ddd, J=8.3, 2.6, 1.5 Hz, ¹H, Ar—H), 7.38 (dd, J=8.3, 4.7 Hz, ¹H, Ar—H), 7.30 (ddd, J=11.6, 8.2, 1.4 Hz, ¹H, Ar—H), 7.21 (t, J=7.3 Hz, ¹H, Ar—H), 7.11-7.06 (m, ¹H, Ar—H). HRMS-ESI m/z [M+H]+calcd for C₁₂H₁₀FN₃O: 232.0808, found: 232.0871.

### EXAMPLE 4

Compound 4 shown below was prepared according to the method of Embodiment (I) which is substituted compound b by using R—Cl

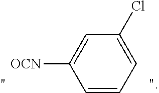

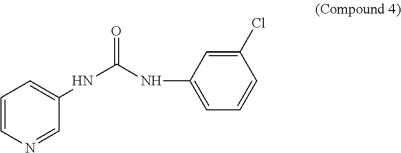 [SMILES: O=C(Nc1cccnc1)Nc1cccc(Cl)c1]

The product represented as Compound 4 of Example 4 is white solid, and yield is 92%. mp: 178.0° C.-178.7° C. ¹H NMR (400 MHz, DMSO) δ 9.32 (br, ¹H, NH), 8 63 (d, J=1.9 Hz, ¹H, Ar—H), 8.19 (d, J=4.5 Hz, ¹H, Ar—H), 7.96-7.93 (m, ¹H, Ar—H), 7.73 (s, ¹H, Ar—H), 7.32-7.26 (m, ³H, Ar—H), 7.03-7.01 (m, ¹H, Ar—H), HRMS-ESI m/z [M+H]+calcd for C₁₂H₉ClN₃O: 248.0512, found: 248.0577.

### EXAMPLE 5

Compound 5 shown below was prepared according to the method of Embodiment (I) which is substituted compound b by using R—Cl

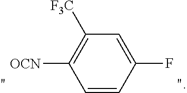

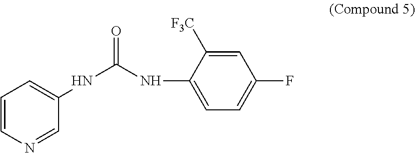 [SMILES: O=C(Nc1cccnc1)Nc1ccc(F)cc1C(F)(F)F]

The product represented as Compound 5 of Example 5 is white solid, and yield is 46% mp: 132.8° C.-134.8° C. ¹H NMR (400 MHz, DMSO) δ 9.53 (s, ¹H NH), 8.61 (d, J=2.5 Hz, ¹H, Ar—H), 8.22-8.21 (m, ²H, Ar—H and NH), 7.98-7.93 (m, ²H, Ar—H), 7.71-7.64 (m, ²H, Ar—H), 7.35-7.29 (m, ²H, Ar—H). HRMS-ESI m/z [M+H]+calcd for C₁₃H₁₀F₃N₃O: 281.0776, found: 282.0838. m.p. 209.5° C.-210.1° C., LC-MS: [M+1]+471.80; ¹H-NMR(CD 3Cl): δ (ppm) 8.65 (s, ¹H, NH), 8.58 (d, ¹H PhH), 7.91-7.87 (m, ²H, PhH), 7.71 (s, ¹H, PhH), 7.61 (dd, ¹H, PhH), 7.28 (t, ²H, PhH), 3.86 (s, ³H, OCH3). ¹³C-NMR: δ (ppm) 164 88. 162.33. 147.75, 146.68, 143.55, 133.48, 133.12, 132.49, 132.18, 130.72, 129.85, 127.67, 125.83, 119.24, 117.78. 105.26, 56.25.

### EXAMPLE 6

Compound 6 shown below was prepared according to the method of Embodiment (I) which is substituted compound b by using R—Cl

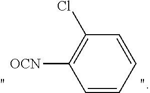

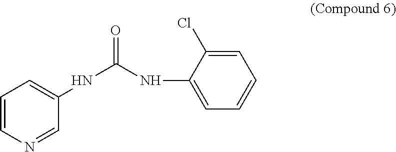 [SMILES: O=C(Nc1cccnc1)Nc1ccccc1Cl]

The product represented as Compound 6 of Example 6 is yellow solid, and yield is 91%.
mp: 185.6° C.-186.6° C. ¹H NMR (400 MHz, DMSO) δ 9.01 (br, 1H, NH), 8.61 (d, J=2.5 Hz, ¹H, Ar—H), 8.21 (dd, J=4.7. 1.4 Hz, ¹H, Ar—H), 8.14 (dd, J=8.3, 1.5 Hz, ¹H, Ar—H), 7.97 (ddd, J=8.3, 2.6, 1.5 Hz, ¹H, Ar—H), 7.47 (dd, J=8.0, 1.4 Hz, ¹H, Ar—H), 7.35-7.29 (m, ²H, Ar—H), 7.06 (td, J=7.7, 1.5 Hz, ¹H, Ar—H). HRMS-ESI m/z [M+H]+calcd for C₁₂H₁₀ClN₃O: 247.0512, found: 248.0576. m.p. 177.3° C.-178.5° C.; LC-MS:[M+1] ⁺458.82 ¹H-NMR (400 MHz, CD₃Cl): δ (ppm) 9.10 (s, ¹H, NH), 8.67 (d, ¹H, PhH), 8.10-8.05 (m, ³H, PhH), 7.89 (dd, ¹H, PhH), 7.62 (d, ¹H, PhH), 7.55 (dd, ¹H, PhH), 7.34-7.23 (m, ³H,PhH), 3.55 (s, ³H, OCH₃), 2.47 (s, ³H, CH₃). ¹³C-NMR (100 MHz, CD₃Cl): δ (ppm) 164.86, 162.24. 147.79, 147.09, 145.94, 143.66, 133.57, 133.10, 132.46. 131.16, 130.82. 129.79, 129.06, 125.74. 125.15, 119.25, 117.84, 105.25, 56.01, 22.09.

### EXAMPLE 7

Compound 7 shown below was prepared according to the method of Embodiment (I) which is substituted compound b by using R—Cl

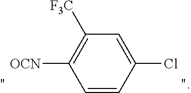

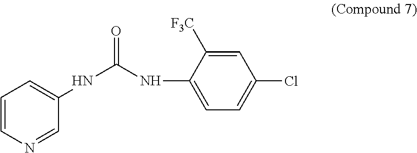 [SMILES: O=C(Nc1cccnc1)Nc1ccc(Cl)cc1C(F)(F)F]

The product represented as Compound 7 of Example 7 is white solid, and yield is 64%. mp: 160.0° C.-162.3° C. ¹H NMR (400 MHz, DMSO) δ 8.62 (d, J=2.5 Hz, ¹H, Ar—H), 8.18 (d, J=4.6 Hz, ¹H, Ar—H), 8.00-7.96 (m, ²H, Ar—H), 7.70-7.67 (m, ²H, Ar—H), 7.30 (dd, J=8.3, 4.7 Hz, ¹H, Ar—H). HRMS-ESI m/z [M+H]+calcd for C₁₃H₉ClF₃N₃O: 315.0386, found: 316.0449.

### EXAMPLE 8

Compound 8 shown below was prepared according to the method of Embodiment (I) which is substituted compound b by using R—Cl

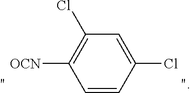

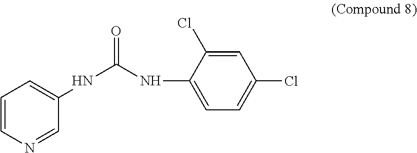 [SMILES: O=C(Nc1cccnc1)Nc1ccc(Cl)cc1Cl]

The product represented as Compound 8 of Example 8 is white solid, and yield is 74%. mp: 186.8° C.-189.0° C. ¹H NMR (400 MHz, DMSO) δ 8.61 (d, J=2.4 Hz, ¹H, Ar—H), 8 22 (dd, J=4,7, 1.4 Hz, ¹H, Ar—H), 8.18 (d, J=9.0 Hz, ¹H, Ar—H), 7.96 (ddd, J=8.3, 2.5, 1.5 Hz, ¹H, Ar—H), 7.63 (d, J=2.4 Hz, ¹H, Ar—H), 7.39 (dd, J=9.0, 2.5 Hz, ¹H, Ar—H), 7.34 (dd, J=8.3, 4.7 Hz, ¹H, Ar—H). HRMS-ESI m/z[M+H]+calcd for C₁₂H₉Cl₂N₃O: 282.0123, found: 282.0188.

### EXAMPLE 9

Compound 9 shown below was prepared according to the method of Embodiment (I) which is substituted compound b by using R—Cl

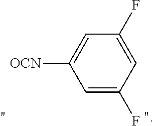

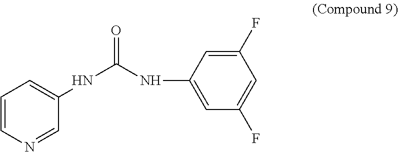 [SMILES: O=C(Nc1cccnc1)Nc1cc(F)cc(F)c1]

The product represented as Compound 9 of Example 9 is white solid, and yield is 52%. m.p. 230.4° C.-231.5° C.; mp: 203.0° C.-205.3° C. ¹H NMR (400 MHz, DMSO) δ 8.62 (d, J=2.5 Hz, ¹H, Ar—H), 8.19 (dd, J=4.7. 1.3 Hz, ¹H, Ar—H), 7.94 (ddd, J=8.3, 2.5. 1.5 Hz, ¹H, Ar—H), 7.30 (dd, J=8.3, 4.7 Hz, ¹H, Ar—H), 7.22 (dd, J=10.0. 2.2 Hz, ²H, Ar—H), 6.77 (tt, J=9.4, 2.3 Hz, ¹H, Ar—H). HRMS-ESI m/z[M+H]+calcd for C₁₂H₉F₂N₃O: 250.0714, found: 250.0777.

### EXAMPLE 10

Compound 10 shown below was prepared according to the method of Embodiment (I) which is substituted compound b by using R—Cl

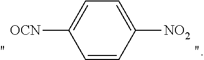

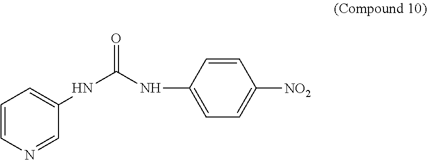 [SMILES: O=C(Nc1ccc([N+](=O)[O-])cc1)Nc1cccnc1]

The product represented as Compound 10 of Example 10 is white solid, and yield is 44%.
m.p. 182.0° C.-183.3° C.; mp: 224.4° C.-226.1° C. ¹H NMR (400 MHz, DMSO) δ 8.68 (d, J=2.5 Hz, ¹H, Ar—H), 8.32 (s, ¹H, NH), 8.18 (dd, J=4.7, 1.4 Hz, ¹H, Ar—H), 8.16-8.12 (m, ²H, Ar—H), 7.98 (ddd, J=8.4, 2.5, 1.5 Hz, ¹H, Ar—H), 7.73-7.69 (m, ²H, Ar—H), 7.30 (dd, J=8.3, 4.6 Hz, ¹H, Ar—H). HRMS-ESI m/z[M+H]+calcd for C₁₂H₁₀N₄O₃: 258.0753, found: 259.0816.

### EXAMPLE 11

Compound 11 shown below was prepared according to the method of Embodiment (I) which is substituted compound b by using R—Cl

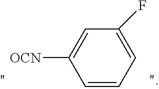

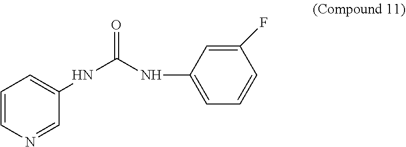 [SMILES: O=C(Nc1cccnc1)Nc1cccc(F)c1]

The product represented as Compound 11 of Example 11 is white solid, and yield is 35%.
m.p. 171.2° C.-172.3° C.; mp: 180.3° C.-181.4° C. ¹H NMR (400 MHz, DMSO) δ 9.04 (br, ²H, NH), 8.61 (d, J=2.5 Hz, ¹H, Ar—H), 8.21 (dd, J=4.7, 1.4 Hz, ¹H, Ar—H), 7.94 (ddd, J=8.3, 2.6, 1.5 Hz, ¹H, Ar—H), 7.49 (dt, J=12.0, 2.3 Hz, ¹H, Ar—H), 7.35-7.29 (m, ²H, Ar—H), 7.15 (dd, J=8.2, 1.2 Hz. ¹H, Ar—H), 6.81 (td, J=8.3. 2.3 Hz, ¹H, Ar—H), HRMS-ESI m/z [M+H]+calcd for C₁₂H₁₀FN₃O: 232.0808, found: 232.0872.

### EXAMPLE 12

Compound 12 shown below was prepared according to the method of Embodiment (I) which is substituted compound b using

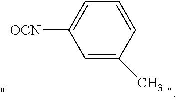

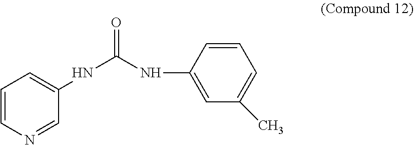 [SMILES: Cc1cccc(NC(=O)Nc2cccnc2)c1]

The product represented as Compound 12 of Example 12 is white solid, and yield is 35%. mp: 182.4° C.-184.1° C. ¹H NMR (400 MHz, DMSO) δ 9.06 (br, ¹H, NH), 8.61 (d, J=2.4 Hz, ¹H, Ar—H), 8.17 (dd, J=4.6, 1.3 Hz, ¹H, Ar—H), 7.94 (ddd, J=8.3, 2.6, 1.5 Hz, ¹H, Ar—H), 7.36 (d, J=8.4 Hz, ²H, Ar—H), 7.30 (dd, J=8.3. 4.7 Hz. ¹H, Ar—H), 7.09 (d, J=8.3 Hz, ²H, Ar—H), 2.25 (s, ³H, CH3), HRMS-ESI m/z[M+H]+calcd for C₁₃H₁₃N₃O: 227.1059, found: 228 1120.

### EXAMPLE 13

Compound 13 shown below was prepared according to the method of Embodiment (I) which is substituted compound b by using

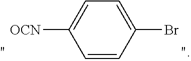

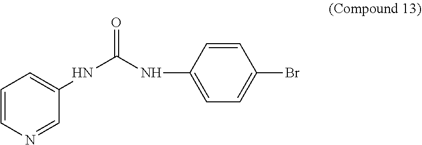 [SMILES: O=C(Nc1ccc(Br)cc1)Nc1cccnc1]

The product represented as Compound 13 of Example 13 is white solid, and yield is 35%
mp: >215° C. ¹H NMR (400 MHz, DMSO) δ 9.05 (br, ¹H, NH), 8.61 (d, J=2.4 Hz, ¹H, Ar—H), 8.19 (dd, J=4.6. 1.3 Hz, ¹H, Ar—H), 7.93 (ddd, J=8.3, 2.5, 1.5 Hz. ¹H, Ar—H), 7.48-7.43 (m, ⁴H, Ar—H), 7.32 (dd, J=8.3, 4.7 Hz, ¹H, Ar—H). HRMS-ESI m/z [M+H]+calcd for C₁₂H₁₀BrN₃O: 291 0007, found 292.0070.

### EXAMPLE 14

Compound 14 shown below was prepared according to the method of Embodiment (I) which is substituted compound b by using R—Cl

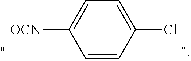

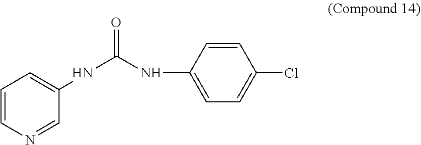 [SMILES: O=C(Nc1ccc(Cl)cc1)Nc1cccnc1]

The product represented as Compound 14 of Example 14 is white solid, and yield is 35%.
m.p. 171.2° C.-172.3° C.; mp: >215° C. ¹H NMR (400 MHz, DMSO) δ 9.19 (br, ¹H, NH), 8.62 (d, J=2.5 Hz, ¹H, Ar—H), 8.19 (d, J=4.6 Hz, ¹H, Ar—H), 7.94 (ddd, J=8.3, 2.5, 1.5 Hz, ¹H, Ar—H), 7.51 (d, J=8.8 Hz, ²H, Ar—H), 7.34-7.29 (m, ³H, Ar—H) HRMS-ESI m/z [M+H]+calcd for C₁₂H₁₀ClN₃O: 247.0512, found: 248.0577.

EXAMPLE 15

Compound 15 shown below was prepared according to the method of Embodiment (I) which is substituted compound b by using

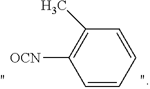

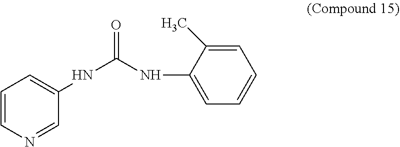 [SMILES: Cc1ccccc1NC(=O)Nc1cccnc1]

The product represented as Compound 15 of Example 15 is white solid, and yield is 35%.
mp: 160.2° C.-160.4° C. ¹H NMR (400 MHz, DMSO) δ 9.22 (br, ¹H, NH), 8.61 (d, J=2.5 Hz, ¹H, Ar—H), 8.18 (dd, J=4.7, 1.4 Hz, ¹H, Ar—H), 7.97 (ddd, J=8.3, 2.6, 1.5 Hz, ¹H, Ar—H), 7.80 (d, J=7.3 Hz, ¹H, Ar—H), 7.32 (dd, J=8.3, 4.6 Hz, ¹H, Ar—H), 7.20-7.14 (m, ²H, Ar—H), 7.00-6.95 (m, ¹H, Ar—H), 2.25 (s, ³H, CH3), HRMS-ESI m/z [M+H]+calcd for C₁₃H₁₃N₃O: 227.1059, found: 228.1122.

### EXAMPLE 16

Compound 16 shown below was prepared according to the method of Embodiment (I) which is substituted compound b by using

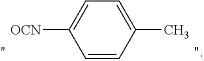

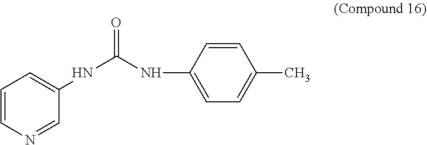 [SMILES: Cc1ccc(NC(=O)Nc2cccnc2)cc1]

The product represented as Compound 16 of Example 16 is white solid, and yield is 35%.
mp: 182.4° C.-184.1°C. ¹H NMR (400 MHz, DMSO) δ 9.06 (br, ¹H, NH), 8.61 (d, J=2.4 Hz, ¹H, Ar—H), 8.17 (dd, J=4.6. 1.3 Hz, ¹H, Ar—H), 7.94 (ddd, J=8.3, 2.6, 1.5 Hz, ¹H, Ar—H), 7.36 (d, J=8.4 Hz, ²H, Ar—H), 7.30 (dd, J=8.3. 4.7 Hz, ¹H, Ar—H), 7.09 (d, J=8.3 Hz, ²H, Ar—H), 2.25 (s, ³H, CH₃). HRMS-ESI m/z[M+H]+calcd for C₁₃H₁₃N₃O: 227.1059, found: 228.1120.

### Embodiment (II): Snail Killing Activity Test and Toxicity Test for Fish of the Compound of the Present Invention

(1): Snail-Killing Activity Against Oncomelania hupensis

Oncomelania hupensis was used as a tested sample and tested by the immersion method.
Operation process: Weigh various samples accurately (compounds of any of Example 1 to Example 16), dissolve each sample with 0.2 mL of N,N-dimethylformamide, and dilute each sample to 0.2 mg/L of the drug solution with dechlorinated tap water. Put 30 snails in each beaker and pour 100 ml of the above-mentioned drug solution into each beaker. And cover the plastic gauze on the beaker to prevent the snails from climbing to the liquid surface. Place the above beakers accommodating the drug solution and snails in the incubator under the conditions which is at 25° C. constant temperature, and the humidity is maintained at 60%. and the light is fully. After 24 hours of immersion in the incubator, the drug solution is poured, and each sample is washed for 3 times by using water. Then, 15 mL of dechlorinated tap water is added to resuscitate each sample for 1 hour. Each sample is resuscitated for 1 hour again after 24 hours, and then activity of the snails is determined by knocking after 24 hours. Each sample was set to repeat 3 times. The tested concentrations were respectively 0.1 mg/L, 0.5 mg/L, 1 mg/L, 2 mg/L,
5 mg/L, and 10 mg/L. A linear interpolation method was used to draw a curve of the percentage of death at the 24^{th} hour relative to the tested concentrations of the prepared compounds, thereby the LC₅₀ values at the 24^{th} hour are obtained.

A comparison was made with 0.2 mg/L of niclosamide (positive control) and water containing 0.2 mL/L of N,N-dimethylformamide (blank control).

(2): Study on Acute Toxicity to Fish

Tested substance: Compound of any of Example 1 to Example 16 of which tested concentrations were respectively 0 mg/L, 20 mg/L, 40 mg/L, 60 mg/L, 80 mg/L, and 100 mg/L.

Tested fish species: Danio rerio of which body length was 20±1 mm and weight was 0.3±0.1 g.

Test process: The tested fish was domesticated in continuous aerated diluted water for 7 days before the test. The water quality and lighting conditions during the domestication were consistent with the conditions during the test. Feeding was stopped 24 hours before the test. The mortality of the tested fish during domestication did not exceed 10%. The water temperature during the test was constant at 23±1° C. The dissolved oxygen content during the test should be higher than 60% of the air saturation value, and the pH value was 7.0±0.2. The test cycle is 96 hours. The poisoning symptoms and mortality of the tested fish were observed and recorded at any time within 3 hours to 6 hours after starting the test, and then the poisoning symptoms and mortality of the tested fish at different concentrations were observed and recorded at 24^{th} hour, 48^{th} hour, 72^{th} hour, and 96^{th} hour. The criterion for judging dead fish is to knock the tail of the fish with glasses. If the fish had no action, it is judged that the fish was dead.

A linear interpolation method was used to draw the curve of the percentage of death relative to the tested concentrations of the compound prepared in Example 4 at 96 hour, thereby the LC₅₀ values at 96^{th} hour are obtained, which was treated with water as a blank control. The LC₅₀ value of the other compound at 96^{th} hour was obtained in this way.

The toxicity results of the compounds prepared in Example 1 to Example 16 on fish are shown in Table 1.

TABLE 1

| | Medical activity | | |
|---|---|---|---|
| | | Oncomelania hupensis | Danio rerio |
| Compound | | the LC₅₀ values at | the LC₅₀ values |
| No | | the 24^{th} hour | at the 96^{th} hour |

| | | | | |
|---|---|---|---|---|
| 1 | 5 | mg/L | >100 | mg/L |
| 2 | 3.5 | mg/L | >100 | mg/L |
| 3 | 4.8 | mg/L | >100 | mg/L |
| 4 | 5.8 | mg/L | >100 | mg/L |
| 5 | 8.9 | mg/L | >100 | mg/L |
| 6 | 9.2 | mg/L | >100 | mg/L |
| 7 | 6.7 | mg/L | >100 | mg/L |
| 8 | 5.8 | mg/L | >100 | mg/L |
| 9 | 4.3 | mg/L | >100 | mg/L |
| 10 | 3.8 | mg/L | >100 | mg/L |
| 11 | 4.9 | mg/L | >100 | mg/L |
| 12 | 8.2 | mg/L | >100 | mg/L |
| 13 | 0.52 | mg/L | 15 | mg/L |
| 14 | 0.53 | mg/L | 15 | mg/L |
| 15 | 7.2 | mg/L | >100 | mg/L |
| 16 | 8.1 | mg/L | >100 | mg/L |
| niclosamide | 0.6 | mg/L | 0.2 | mg/L |

| | | |
|---|---|---|
| water | — | — |

The result is shown below:

1) The snail-killing effects of the compounds of the present invention are all excellent, and the snail-killing effect of most compounds at 10 mg/L has reached 100%. Wherein LC₅₀ values of compounds 13 and 14 are about 0.5 mg/L which are higher than the snail-killing activity of niclosamide.
2) The LC₅₀ values of the compounds of the present invention for fish at 96^{th} hour were all greater than 10 mg/L. When the LC₅₀ value of niclosamide at 96^{th} hour is 0.21 mg/L and was applied for 1 hour, all fish is resulted in almost death. It can be seen that the compounds of the present invention have very low toxicity to fish. Therefore, it is safe for the environment.

### Embodiment (III): Preparation of a Molluscicide Composition Containing the Compound of the Present Invention

1. Preparation of molluscicide solution:

The active compound (the compound prepared in any of Example 1 to Example 16) was accurately weighed, dissolved in 0.2 mL of N,N-dimethylformamide, and diluted to a drug solution of 0.5 mg/L with dechlorinated tap water.

2. Preparation of anti-snail granules:

The active compound (the compound prepared in any of Example 1 to Example 16) was accurately weighed, and mixed with a suitable filler. Dry sand or chemical fertilizer may be used as the filler when diluting. Mixing in a certain ratio is well.

All documents mentioned in the present invention are incorporated by reference in this application, as if each document was individually incorporated by reference. In addition, it should be understood that after reading the above-mentioned teaching content of the present invention, those skilled in the art can make various changes or modifications to the present invention, and these equivalent forms also fall within the scope defined by the claims attached to this application.

1. A compound having a structure of formula (I), an optical isomer thereof, a racemate thereof, a solvate thereof, or a pharmaceutically acceptable salt thereof: 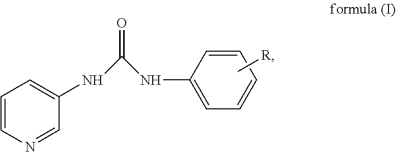
in formula (I), substituent R is selected from the group consisting of p-methoxy, fluorine, chlorine, bromine, trifluoromethyl, methyl, nitro and hydrogen.

2. A use of a compound of formula (I), an optical isomer thereof, a racemate thereof, a solvate thereof, or a pharmaceutically acceptable salt thereof, for the preparation of an agricultural composition or a formulation, the agricultural composition or formulation is for (a) preventing parasites, and/or (b) killing a parasitic vector, wherein the parasitic vector is various snails, 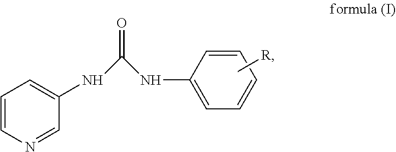
in formula (I), substituent R is selected from the group consisting of p-methoxy, fluorine, chlorine, bromine, trifluoromethyl, methyl, nitro and hydrogen.

3. The application according to claim 2, wherein the parasite comprises nematode, tapeworm, and trematode.

4. The application according to claim 2, wherein the parasitic vector comprises Oncomelania hupensis, Pomacea canaliculata, and snail.

5. An agricultural composition comprising:
(a) an active component which is a compound of formula (I), an optical isomer thereof, a racemate thereof, a solvate thereof, or a pharmaceutically acceptable salt thereof: 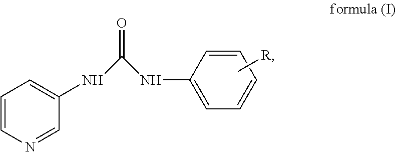
in formula (I), substituent R is selected from the group consisting of p-methoxy, fluorine, chlorine, bromine, trifluoromethyl, methyl, nitro and hydrogen, and (b) an agrochemically acceptable carrier or excipient.

6. The agricultural composition according to claim 5, wherein the agricultural composition contains from 0.0001 wt % to 99.99 wt % of compound of formula (I), an optical isomer thereof, a racemate thereof, a solvate thereof, or a pharmaceutically acceptable salt thereof, based on the total weight of the agricultural composition.

7. The agricultural composition according to claim 5, wherein the agricultural composition further comprises other molluscicide, the other molluscicide is commercially available.

8. The agricultural composition according to claim 7, wherein the other molluscicide is selected from the group consisting of niclosamide. Rongbao, Rongya, tee tree seed and other commercially available plant molluscicides.

9-11. (canceled)

12. The agricultural composition according to claim 5, wherein agricultural composition is applied at a concentration from 0.02 mg/L to 5 mg/L.
